(19) United States
(12) Patent Application Publication
(10) Pub. No.: US 2023/0320740 A1
(43) Pub. Date: Oct. 12, 2023

(54) Tongue Cleaner

(71) Applicant: Jamilla Hemeid, Montclair, NJ (US)

(72) Inventor: Jamilla Hemeid, Montclair, NJ (US)

(21) Appl. No.: 18/060,219

(22) Filed: Nov. 30, 2022

Related U.S. Application Data
(63) Continuation of application No. 29/833,863, filed on Apr. 7, 2022.

(51) Int. Cl. A61B 17/24; A46B 15/00

(52) U.S. Cl. CPC A61B 17/244; A46B 15/0081

(57) ABSTRACT

A tongue cleaner is disclosed includes a lower holding portion and an upper scraping portion, wherein the lower holding portion includes a pair of laterally opposing side portions formed from a first side portion and a second side portion, and a pair of laterally opposing end portions formed from a first end portion and a second end portion, where the upper scraping portion includes a first leg portion, a second leg portion, a scraping surface, and a closed-loop opening bounded by the first leg portion, the second leg portion, the scraping surface, and the second end portion of the lower holding portion, and where the first and second leg portions extend from the second end portion of the lower holding portion, and the upper scraping surface includes a first, honed edge and a second, smoothed edge.

### CROSS-REFERENCE TO RELATED APPLICATIONS

This application claims priority from U.S. Design application Ser. No. 29/833,863 filed Apr. 7, 2022, the content of which is incorporated herein by reference in its entirety.

### BACKGROUND OF THE INVENTION

Modern dental practice has determined that it is necessary and essential to clean not only the teeth but also the tongue to properly clean the month and to reduce the incidence of bad breath. Due to the non-uniform surface of the tongue, it may be a breeding ground for microorganisms such as bacteria, tartar, and may retain food debris. The debris and/or plaque that build up on the tongue are related to many common oral care issues and general health problems. In addition, decaying bacteria may produce volatile chemical compounds on the rear part of the tongue, which may account for bad breath. Scientific researchers have shown that routine tongue cleaning/scraping may eliminate much of the undesirable coating on the tongue, and thus may significantly improve overall oral hygiene.

However, cleaning/scraping of the tongue has often been overlooked, or alternatively has been attempted in a primitive manner via a conventional toothbrush. The toothbrush, however, is not well suited for this purpose because the bristles of the toothbrush are often too soft and flexible to remove the above-described hard buildup. In addition, the overall size, shape, and configuration of the toothbrush usually make the toothbrush poorly suited for tongue cleaning/scraping.

Thus, various techniques have been developed specifically for effecting tongue cleaning. For example, a tongue cleaner/scraper, a tongue brusher, or other similar oral hygiene devices have been invented to clean the bacterial build-up and food debris from the surface of the tongue.

A wide variety of tongue cleaners are known in the dental hygiene arts. Some tongue cleaners incorporate rigid blade-like structures while others may be flexible, or have brush-like structures. Also known are tongue cleaners that are both reusable and disposable. Some tongue cleaners may also include flavorings or fragrances. Generally, tongue cleaners are fabricated of a wide variety of materials, e.g., natural and synthetic, and are designed in a wide variety of shapes and constructions.

U.S. Pat. No. 3,890,964 discloses a tongue cleaner including an elongated handle with longitudinally diverging arms, where the ends of the arms are interconnected by a longitudinally bowed cross-bar configured for cleaning the tongue. Various types of tongue cleaners for cleaning/scraping the tongue to remove materials accumulated thereon are known from U.S. Pat. Nos. 3,943,592, 4,079,478, 5,735,864, and 6,171,323.

### SUMMARY OF THE INVENTION

Disclosed is an improved oral-hygiene device or a tongue cleaner, which is used to remove plaque, tartar, bacteria, and food debris from the tongue. Specifically, the disclosed device is a reusable tongue cleaner with a unique shape and configuration, that is reusable, sterilizable and/or easily rinsed, and that effectively and harmlessly cleans the tongue to promote the reduction or elimination of bad breath, tooth decay, gum problems, and other periodontal diseases.

The discussed cleaning device has several advantages over existing tongue cleaners, such as a unique dual cleaning mode comprising a sharp scraping edge as well as a smooth scraping edge for different cleaning needs. The inventive cleaning device also has an integral rigid structure that permits it to stand on a flat surface and allows complete control of the device in desired areas of the tongue. Further, the inventive tongue cleaning device has a sleek, aerodynamic appearance configured to be psychologically comforting by resembling common eating utensils, as opposed to other razor shaped, bulky designs of conventional tongue cleaners. These features may not be found in traditional flimsy, plastic tongue cleaners. Accordingly, the disclosed improved tongue cleaner provides a simplified, effective, safe, inexpensive, and efficient structure in comparison to existing tongue cleaning devices.

The herein-described embodiments address these and other problems associated with the art by providing in one aspect a tongue cleaner including a lower holding portion and an upper scraping portion. The lower holding portion includes a pair of laterally opposing side portions formed from a first side portion and a second side portion, and a pair of laterally opposing end portions formed from a first end portion and a second end portion. The upper scraping portion includes a first leg portion, a second leg portion, a scraping surface, and a closed-loop opening bounded by the first leg portion, the second leg portion, the scraping surface, and the second end portion of the lower holding portion. The first and second leg portions extend from the second end portion of the lower holding portion, and the upper scraping surface includes a first, honed edge and a second, smoothed edge.

In certain embodiments, the lower holding portion is formed in an elongated manner and includes a pair of laterally opposing outside surfaces having a first outside surface and a second outside surface, and the first and second outside surfaces are curved/rounded. In such embodiments, the first and second side portions may also be curved/rounded. In some embodiments, the first end of the lower holding portion has a standing base configuration, and the first end of the lower holding portion has a slip-resistant base. In some embodiments, the first end of the lower holding portion has a recessed bottom.

In certain embodiments, the closed-loop opening of the upper scraping portion has a parabolic shape, and the first leg portion and the second leg portion may extend in a “Y”-shaped manner with the scraping surface extending in a curved/arcuate configuration therebetween. In alternative embodiments, the first, honed scraping edge may have a plurality of massaging teeth and/or a bristle brush configuration. In other embodiments, the tongue cleaner is formed from a nonporous, rigid, and fracture resistant material. In such embodiments, the nonporous, rigid, and fracture resistant material may be stainless steel and/or a polymer material. In certain embodiments, the lower holding portion and the upper scraping portion may be made of different materials. In certain embodiments, the lower holding portion and the upper scraping portion may also have different surface finishes.

Other objects and features of the present invention will become apparent from the following detailed description considered in conjunction with the accompanying drawings. It is to be understood, however, that the drawings are designed solely for purposes of illustration and not as a definition of the limits of the invention. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

An exemplary embodiment of the invention is explained in greater detail below with reference to the drawings, this comprising schematic and greatly simplified figures in which:

FIG. 1 is a perspective view of a tongue cleaner, in accordance with an embodiment of the invention;

FIGS. 2a-e are illustrations of the front, side, back, top, and bottom plan views of the tongue cleaner of FIG. 1;

FIG. 3 is a sectional front view of the scraping portion of the tongue cleaner of FIG. 1; and

FIG. 4 is a rear view of the scraping portion of the tongue cleaner of FIG. 1.

### DETAILED DESCRIPTION OF THE EXEMPLARY EMBODIMENTS

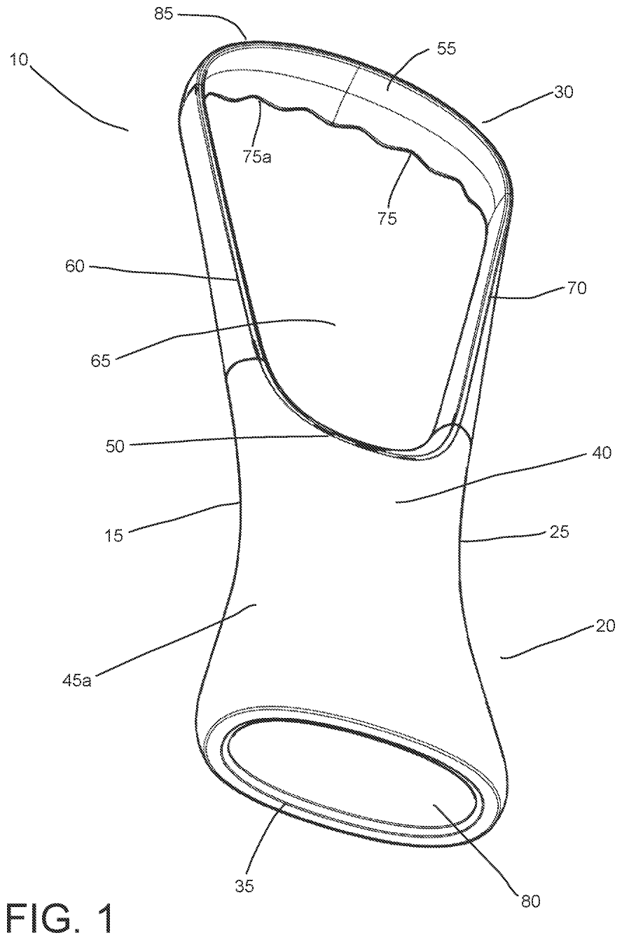

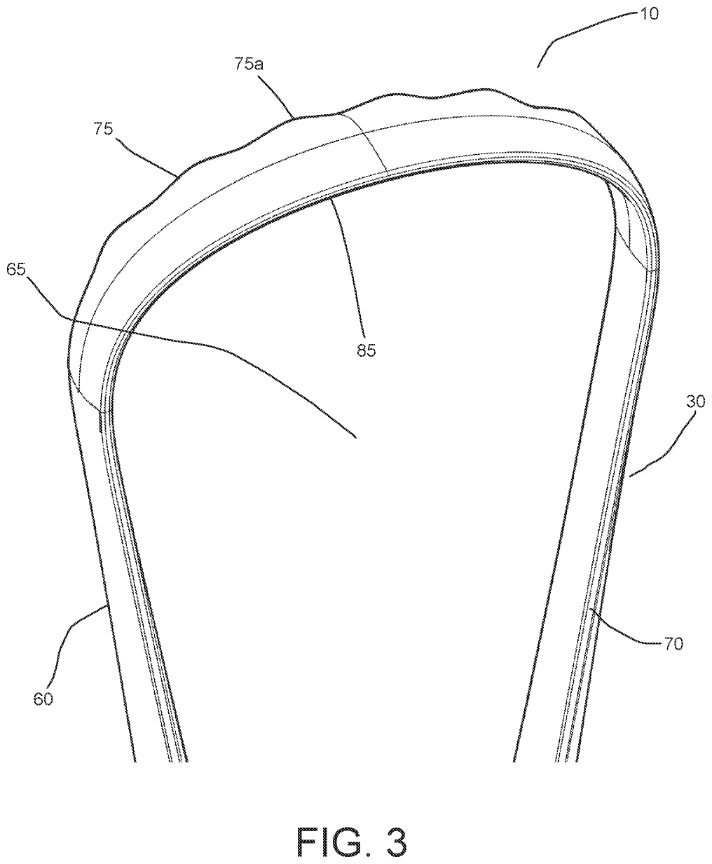

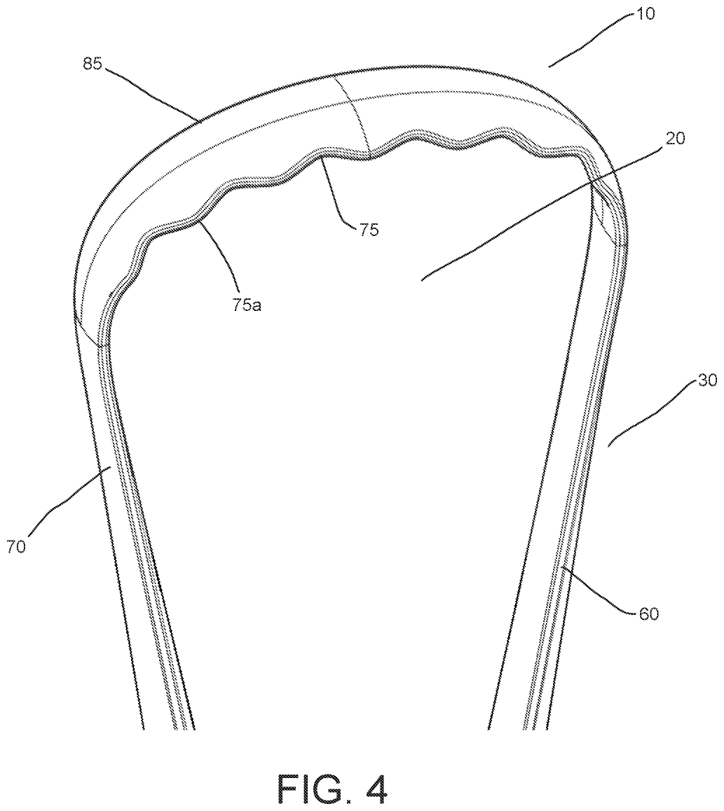

As shown in FIG. 1 through FIG. 4, a tongue cleaner 10 in accordance with the invention includes a lower section that forms a lower holding portion 20 and an upper section that forms a scraping portion 30. In certain embodiments, the lower holding portion 20 may have an elongated body structure 40 with a pair of laterally opposing side portions including a first side portion 15 and a second side portion 25 and a pair of laterally opposing end portions including a first end portion 35 and a second end portion 50. In certain embodiments, the second end portion 50 of the lower holding portion 20 terminates by splitting/diverging in a “Y-like” manner into the upper scrapping portion 30, thereby forming a first leg portion 60 and a second leg portion 70. Specifically, the pair of laterally opposing leg portions 60 and 70 may each split off substantially longitudinally and can be in a substantially planar manner from its respective side portions 15 and 25 of the lower holding portion 12. In addition, a scraping surface 55 of the upper scraping portion 30 may transversely extend between the leg portions 60 and 70 to form a closed-loop opening 65 defined/enclosed by the second end 50 of the lower holding portion 20, the pair of leg portions 60 and 70, and the scraping surface 55.

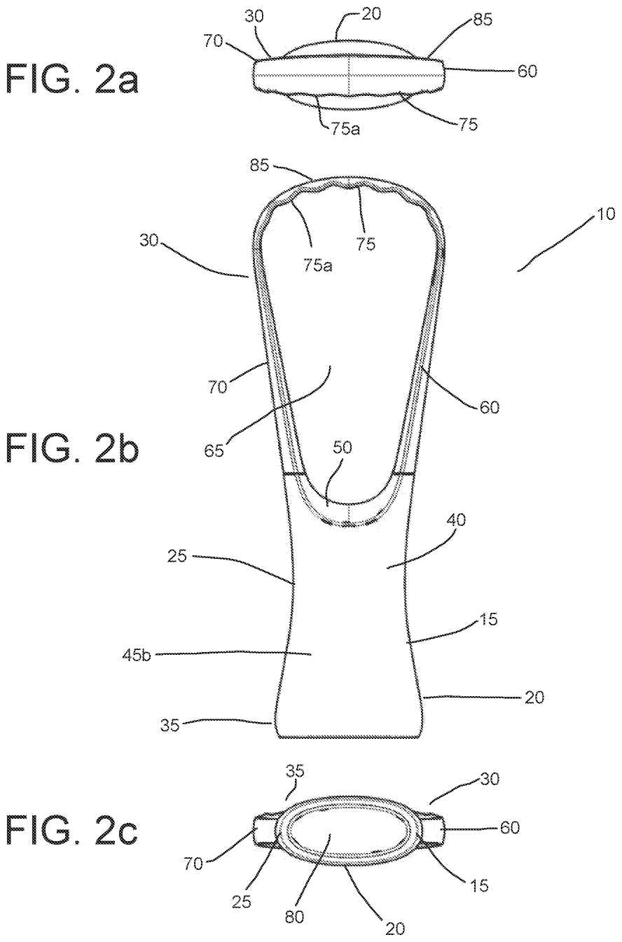

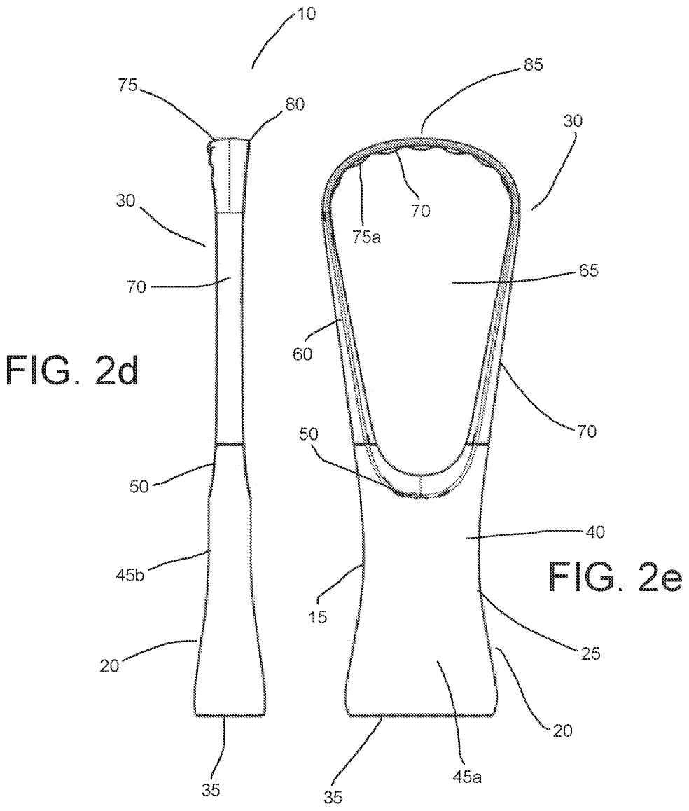

In certain embodiments as shown here, the elongated body structure 40 comprises a pair of rounded/curved laterally opposing outside surfaces including a first outside surface 45a and a second outside surface 45b, with the first and second side portions 15 and 25 that fit within the user's hand that is easy for the user to hold. In certain embodiments, the first end portion 35 is formed as a base stand (e.g., with a flat bottom) so that the tongue cleaner 10 can freely stand via its first end portion 35 on a flat surface upright (e.g., a bathroom counter), without the need for an accessory (e.g., a holder) or placing it to a drawer or a jar. In such embodiment, a slip-resistant base (e.g., a rubber washer/ring) may be provided to the bottom of the first end portion 35 to facilitate the self-standing feature. In some other embodiments, the first end 35 of the lower holding portion 20 may have a recessed bottom 80 as best shown in FIGS. 1 and 2c to minimize the overall weight of the device so that the user could operate it more easily.

In alternative embodiments, the scraping surface 55 includes various scraping edges (e.g., two or more scraping edges formed in different scraping configurations). For example, as best shown in FIGS. 1, 2a, 2b, 2d, 2e, 3 and 4, the scraping surface 55 may have a first, honed edge 75 and a second, smoothed edge 85. In other embodiments, the first, honed edge 75 is alternatively formed in a jagged, toothed, serrated, grooved, notched, stepped, or otherwise patterned or uneven manner including triangular and square toothed configurations. In certain exemplary embodiments, the first honed edge 75 may be formed over the majority of the length of the scraping surface 55 with an array/serrations of scraping teeth 75a. In such embodiments, the scraping teeth 75a are shown as a plurality of arcuate undulations, wavy lines in FIGS. 1-4, and each tooth 75a extends essentially to a tip or high point. The various scraping edges such as the scraping edges 75 and 85 may alternatively be formed from differing constructions/configurations so as to effectively remove the germs, bacteria, microorganisms, food, microorganisms, and other microbial material that may collect, breed, and/or grow, and in some cases turn into plaque and tartar when mixed with saliva while not scraping, cutting or otherwise damaging the tongue or mouth surfaces. In other exemplary embodiments, the first, honed edge 75 may have a hard/stiff bristle brush configuration (not shown here).

During use, the scraping surface 55 may be substantially vertical, while the lower holding portion 65 may be substantially horizontal, to the tongue. In certain embodiments, the closed-loop opening 65 may have a parabolic shape, and the scraping surface 55 extending between the pair of leg portions 60 and 70 may be have a curved/arcuate configuration as shown here. In such embodiments, the curved scraping surface 55 may be configured to fit the muscle contours of the tongue most effectively while the tongue cleaner 10 is dragged with moderate pressure over the tongue surface. In such a manner, the upper scraping portion 30 is ergonomically configured to clean the curves of the tongue and hard to reach places with the mouth.

In other embodiments, the lower holding portion 20 has a length of about 40 mm from the outermost or distal first end portion 35 to the innermost or proximate second end portion 50 at the split where the upper scraping portion 30 starts, a width of about 27 mm from the first side portion 15 to the second side portion 25, and a thickness of about 12 mm from the first outside surface face 45a to the second outside surface face 45b. In alternative embodiments, the upper scraping portion 30 has a length of about 60 mm, such that the overall length of the tongue cleaner 10 may be approximately 100 mm, although it may vary therefrom. In other embodiments, the recessed bottom 80 in the first end 35 of the lower holding portion 20 has a hemispherical configuration, with a height of about 6 mm. It should be understood that a wide range of dimensions is acceptable here, while the length of the disclosed tongue cleaner 10 and its inherent rigid properties make the tongue scraper substantially less likely to be swallowed or inhaled than other short, flimsy products.

The tongue cleaner 10 may be made of any material sufficient to withstand usage requirements while also being sterilizable. In other embodiments, the tongue cleaner 10 is made of a nonporous, rigid, and fracture resistant material. For example, in alternative embodiments, the nonporous, rigid, and fracture resistant material is surgical quality stainless steel, which will not fracture and can be sterilized under intense heat in a repetitive nature without any damage to the inventive device. The stainless steel has a nonporous finish that prevents organic material from collecting on the device. Stainless steel also advantageously provides the entire device and its scraping edges an exceptional level of durability not found in other plastic cleaning tools. For example, the stainless steel construction provides the scraping surface 55 and the scraping edges 75 and 85 with exceptional durability, so that they remain unworn with continued use over time. In particular, the scraping edges 75 and 85 are not easily marred by rough handling. Thus, the stainless steel construction provides a rigid device that is not flimsy, bendable, or otherwise flexible during use under pressure against the tongue of a user. Another advantage of constructing the device from the stainless steel is that the properties of stainless steel allow for unlimited sterilization cycles. For example, the tongue cleaner 10 may be sterilized using a dishwasher or other professional disinfecting machinery. In other embodiments, the nonporous, rigid, and fracture resistant material may be a polymer material with the similar characteristics as stainless steel.

In certain embodiments, the lower holding portion 20 may be made of different materials than that of the upper scraping portion 30. For example, the lower holding portion 20 may be made of any smooth, easy to handle, and user friendly material, besides stainless steel, which is ergonomically shapeable as best shown in FIGS. 2a, 2b, 2d, and 2e. One such material is a polymer material that has a user friendly feel and is replete of any sharp edges or corners that could harm the inner surface of the mouth or harbor germs, buildup, etc. The polymer material is also easily molded into the desired shape. The polymer may be capable of repetitive intense heat during sterilization. While the tongue cleaner 10 is preferably finished to provide a better user experience, the lower holding portion 20 and the upper scraping portion 30 may have different surface finishes in alternative embodiments. For example, the lower holding portion 20 may have a more abrasive surface to increase friction for a better user operation experience (i.e., better grip), while the upper scraping portion 30 may have a high gloss polished surface to prevent users from scratching/hurting their mouth.

In summary, the size and construction of the tongue cleaner 10 may not be limited as long as it can be easily held and operated by users to clean their tongues. In such a manner, the tongue cleaner 10 may be inserted into the mouth with its upper scraping portion 30, specifically the scraping surface 55, in contact with the tongue of the user, while the lower holding portion 20 may then be pulled or moved back and forth by the user to move the upper scraping portion 30 across the tongue to scrape harmful materials and/or debris therefrom.

Thus, while there have been shown, described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements or structure shown which perform substantially the same function in substantially the same way to achieve the same results are within the scope of the invention. Moreover, it should be recognized that elements or structure shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A tongue cleaner, comprising:
a lower holding portion including a pair of laterally opposing side portions formed from a first side portion and a second side portion, and a pair of laterally opposing end portions formed from a first end portion and a second end portion; and
an upper scraping portion including a first leg portion and a second leg portion extending from said second end portion of said lower holding portion, a scraping surface, and a closed-loop opening bounded by the first leg portion, the second leg portion, the scraping surface, and the second end portion of the lower holding portion, said upper scraping surface including a first, honed edge and a second, smoothed edge.

2. The tongue cleaner of claim 1, wherein said lower holding portion is formed in an elongated manner and includes a pair of laterally opposing outside surfaces having a first outside surface and a second outside surface, said first and second outside surfaces being curved/rounded.

3. The tongue cleaner of claim 2, wherein said first and second side portions are curved/rounded.

4. The tongue cleaner of claim 1, wherein said first end of said lower holding portion has a standing base configuration.

5. The tongue cleaner of claim 4, wherein said first end of said lower holding portion has a slip-resistant base.

6. The tongue cleaner of claim 1, wherein said first end of said lower holding portion has a recessed bottom.

7. The tongue cleaner of claim 1, wherein said closed-loop opening of said upper scraping portion has a parabolic shape; and wherein said first leg portion and said second leg portion extend in a “Y”-shaped manner and said scraping surface extends in a curved/arcuate configuration therebetween.

8. The tongue cleaner of claim 1, wherein said first, honed scraping edge has a plurality of massaging teeth.

9. The tongue cleaner of claim 1, wherein said first, honed scraping edge has a bristle brush configuration.

10. The tongue cleaner of claim 1 is formed from a nonporous, rigid, and fracture resistant material.

11. The tongue cleaner of claim 10, wherein said nonporous, rigid, and fracture resistant material is stainless steel.

12. The tongue cleaner of claim 10, wherein said nonporous, rigid, and fracture resistant material is a polymer material.

13. The tongue cleaner of claim 1, wherein said lower holding portion and said upper scraping portion are made of different materials.

14. The tongue cleaner of claim 1, wherein said lower holding portion and said upper scraping portion have different surface finishes.
